(19) United States
(12) Patent Application Publication
(10) Pub. No.: US 2011/0250157 A1
(43) Pub. Date: Oct. 13, 2011

(54) Skin Hyperpigmentation Acyl Glutathione Treatments

(76) Inventor: Nicholas V. Perricone, Meriden, CT (US)

(21) Appl. No.: 13/072,326

(22) Filed: Mar. 25, 2011

Related U.S. Application Data
(63) Continuation-in-part of application No. 12/647,629, filed on Dec. 28, 2009.

(51) Int. Cl. A61K 8/64; A61Q 19/02

(52) U.S. Cl. 424/62

(57) ABSTRACT

Topical compositions for treatment of hyperpigmentation comprise an effective amount of S-acyl glutathione derivative and a carrier. Methods for treating hyperpigmentation, such as melasma, postinflammatory hyperpigmentation and lentigines, skin lightening and skin whitening comprise applying a composition containing S-acyl glutathione derivative in a dermatologically acceptable carrier to skin tissue. The acyl group is a saturated or unsaturated aliphatic C₁₂-C₂₄ group, preferably a C₁₆-C₂₄ group, most preferably a C₁₆ group. In particularly preferred embodiments, the S-acyl glutathione derivative is S-palmitoyl glutathione.

### CROSS-REFERENCE TO RELATED APPLICATIONS

This application is a continuation-in-part of pending U.S. patent application Ser. No. 12/647,629 filed Dec. 28, 2009, entitled Topical Acyl Glutathione Formulations, the content of which is incorporated herein by reference.

### FIELD OF THE INVENTION

The present invention relates to topical compositions comprising glutathione derivatives. More specifically, the present invention relates to topical compositions comprising acyl derivatives of glutathione to treat hyperpigmentation of the skin.

### BACKGROUND OF THE INVENTION

Reduced glutathione, most commonly called glutathione or GSH, is a relatively small molecule found in animals and plants, having the following formula:

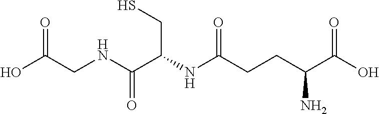

Glutathione is a water-phase orthomolecule. It is the smallest intracellular thiol molecule. It is a potent reducing compound due to its significant electron-donating capacity. Glutathione is a potent antioxidant and enzyme cofactor which plays a critical role in regulating cell activity.

Glutathione is a linear tripeptide of L-glutamine, L-cysteine, and glycine. Technically, N-L-gamma-glutamyl-cysteinyl glycine or L-glutathione, the molecule has a sulfhydryl (SH) group on the cysteinyl portion, which accounts for its strong electron-donating character. As electrons are lost, the molecule becomes oxidized, and two oxidized glutathione molecules become linked (dimerized) by a disulfide bridge to form glutathione disulfide or oxidized glutathione (GSSG). This linkage is reversible upon re-reduction. Glutathione is under tight homeostatic control both intracellularly and extracellularly. A dynamic balance is maintained between glutathione synthesis, its recycling from GSSG/oxidized glutathione, and its utilization.

Glutathione synthesis involves two closely linked, enzymatically controlled reactions that utilize ATP. First cysteine and glutamate are combined by gamma-glutamyl cysteinyl synthetase. Second, glutathione synthetase combines gamma-glutamylcysteine with glycine to generate glutathione. As glutathione levels rise, they self-limit further glutathione synthesis; otherwise, cysteine availability is usually rate-limiting. Fasting, protein-energy malnutrition, or other dietary amino acid deficiencies limit glutathione synthesis.

Glutathione recycling is catalyzed by glutathione disulfide reductase, which uses reducing equivalents from NADPH to reconvert GSSG to 2GSH. The reducing power of ascorbate helps conserve systemic glutathione. Glutathione is used as a cofactor by (1) multiple peroxidase enzymes, to detoxify peroxides generated from oxygen radical attack on biological molecules; (2) transhydrogenases, to reduce oxidized centers on DNA, proteins, and other biomolecules; and (3) glutathione S-transferases (GST) to conjugate glutathione with endogenous substances (e.g., estrogens) and to exogenous electrophiles (e.g., arene oxides, unsaturated carbonyls, organic halides), and diverse xenobiotics.

Free radical and other oxidative agents can deplete glutathione. The homeostatic glutathione redox cycle attempts to maintain glutathione levels as it is being consumed. Amounts available from foods are limited (less than 150 mg/day), and oxidative depletion can outpace synthesis.

The liver is the largest glutathione reservoir. The parenchymal cells synthesize glutathione for P450 conjugation and numerous other metabolic requirements, then export glutathione as a systemic source of SH/reducing power. Glutathione is carried in the bile to the intestinal luminal compartment. Epithelial tissues of the kidney tubules, intestinal lining, and lung, have substantial P450 activity and modest capacity to export glutathione.

Glutathione equivalents circulate in the blood predominantly as cystine, the oxidized and more stable form of cysteine. Cells import cystine from the blood, reconvert it to cysteine (likely using ascorbate as cofactor), and from it synthesize glutathione. Conversely, inside the cell glutathione helps re-reduce oxidized forms of other antioxidants such as ascorbate and alpha-tocopherol.

Glutathione is an extremely important cell protectant. It directly quenches reactive hydroxyl free radicals, other oxygen-centered free radicals, and radical centers on DNA and other biomolecules. Glutathione protects skin, lens, cornea, and retina against radiation damage, and the biochemical foundation of P450 detoxication in the liver, kidneys, lungs, intestinal epithelia, and other organs.

Glutathione is the essential cofactor for many enzymes which require thiol-reducing equivalents, and helps keep redox-sensitive active sites on enzymes in the necessary reduced state. Higher-order thiol cell systems—the metallothioneins, thioredoxins, and other redox regulator proteins—are ultimately regulated by GSH levels and the GSH/GSSG redox ratio.

Glutathione and its metabolites also interface with energetics and neurotransmitter syntheses, through several prominent metabolic pathways. Glutathione availability down-regulates the pro-inflammatory potential of leukotrienes and other eicosanoids.

Glutathione levels in human tissues normally range from 0.1 to 10 millimolar (mM), most concentrated in the liver (up to 10 mM) and in the spleen, kidney, lens, erythrocytes, and leukocytes. Plasma concentration is in the micromolar range (approx. 4.5 μM). Oxidative stressors that can deplete glutathione include ultraviolet and other radiation; viral infections; environmental toxins, household chemicals, and heavy metals; surgery, inflammation, burns, septic shock; and dietary deficiencies of glutathione precursors and enzyme cofactors.

Topical uses of glutathione derivatives have been disclosed. U.S. Pat. No. 3,984,569 (Kalopissis) discloses use of S-substituted linear and branched alkyl and alkenyl derivatives of glutathione for various scalp and hair applications and to combat excessive sebum secretion. U.S. Pat. No. 5,516,507 (N'Guyen) discloses gluathione mono-alkyl esters for topical treatment of cutaneous aging. These glutathione mono-alkyl esters are substituted at the glycine residue and employ alkyl chains having only 1 to 10 carbons. U.S. Pat. App. 2004/0147452 (Yu) proposes the use of non-amphoteric N-acyl glutathione derivatives for topical application for a broad range of conditions. The non-amphoteric derivatives of glutathione are proposed due to the instability of aqueous pharmaceutical formulations of mono and diester prodrugs of glutathione, which rapidly deteriorate over time.

Oral administration of 500 mg per day of glutathione has been reported to result in a lightening of skin color in a small number of subjects. Arjinpathana et al., J Dermatolog Treat 2010 Jun. 5. Also, L-glutathione containing soaps have been promoted as providing skin whitening effects.

It is desired to have improved compositions and methods comprising glutathione derivatives to treat skin hyperpigmentation and to provide improved skin whitening effects relative to the prior art.

### SUMMARY OF THE INVENTION

Accordingly, an object of the present invention is to provide a topical composition for treatment of hyperpigmentation, comprising an effective amount of S-acyl glutathione derivative of formula (I)

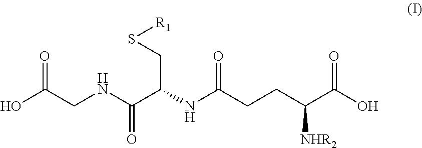

wherein R₁ consists of a saturated or unsaturated aliphatic C₁₂-C₂₄ group and R₂ is a hydrogen, aliphatic or aromatic acyl group; and a dermatologically acceptable carrier.

In some embodiments, R₁ is a C₁₆-C₂₀ group. In some of these embodiments, R₁ is a palmitoyl group.

In certain embodiments, the composition comprises from about 0.01% to about 20% by weight of S-acyl glutathione derivative. In some of these embodiments, the composition comprises from about 0.1% to about 10% by weight of S-acyl glutathione derivative. In certain of these embodiments, the composition comprises from about 3.0% to about 9.0% by weight S-acyl glutathione derivative.

In some embodiments, the carrier comprises fatty acid derivatives of stearic acid.

In certain embodiments, the composition further comprises one or more additional ingredients selected from the group consisting of ascorbic acid and ascorbic acid derivatives, lipoic acid, α-hydroxy acids, and salts of magnesium, zinc and copper, and mixtures thereof.

The invention also provides a method for the treatment of hyperpigmentation comprising applying to skin tissue a composition containing S-acyl glutathione derivative of formula (I)

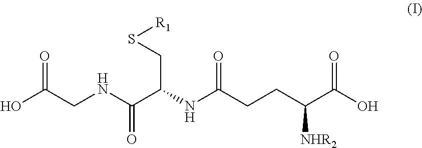

wherein R₁ consists of a saturated or unsaturated aliphatic C₁₂-C₂₄ group and R₂ is a hydrogen, aliphatic or aromatic acyl group; and a dermatologically acceptable carrier.

In some embodiments of the method, R₁ is a C₁₆-C₂₀ group. In some of these embodiments, R₁ is a palmitoyl group.

In certain embodiments, the composition applied to the skin comprises from about 0.01% to about 20% by weight of S-acyl glutathione derivative. In some of these embodiments, the composition comprises from about 0.1% to about 10% by weight of S-acyl glutathione derivative. In certain of these embodiments, the composition comprises from about 3.0% to about 9.0% by weight of S-acyl glutathione derivative.

In some embodiments, the carrier applied to the skin comprises fatty acid derivatives of stearic acid.

In certain embodiments, the composition applied to the skin further comprises one or more additional ingredients selected from the group consisting of ascorbic acid and ascorbic acid derivatives, lipoic acid, α-hydroxy acids, and salts of magnesium, zinc and copper, and mixtures thereof.

In some embodiments the hyperpigmentation treated comprises facial hyperpigmentation. In certain embodiments, the hyperpigmentation treated comprises melasma. In other embodiments, the hyperpigmentation treated comprises postinflammatory hyperpigmentation. In further yet embodiments, the hyperpigmentation treated comprises lentigines.

The invention also provides a method for skin lightening comprising applying a composition containing S-palmitoyl glutathione and a dermatologically acceptable carrier to skin tissue.

The invention further provides a method for skin whitening comprising applying a composition containing S-palmitoyl glutathione and a dermatologically acceptable carrier to skin tissue.

### DETAILED DESCRIPTION OF THE INVENTION

The present invention comprises topical S-acyl glutathione (GSH) compositions to treat hyperpigmentation of the skin. These compositions may also be referred to using IUPAC nomenclature as S-alkanoyl glutathione compositions. The treatments consist of S-acyl glutathione derivatives of the formula:

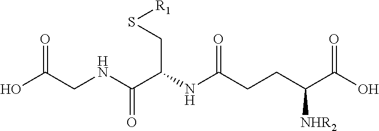

wherein R₁ is consists of a saturated or unsaturated aliphatic C₁₂-C₂₄ group, preferably a C₁₆-C₂₄ group, most preferably a C₁₆ group; and R₂ is a hydrogen, aliphatic or aromatic acyl group, and most preferably a hydrogen group. In preferred embodiments, R₁ is selected from the group consisting of linoleoyl, oleoyl or palmitoyl groups, but is most preferably a palmitoyl group. A particularly preferred embodiment of the invention comprises S-palmitoyl glutathione.

Hyperpigmentation is caused by an increase in melanin. It may occur in the skin or nails. The condition may be acquired, congenital or inherited. Pigmentation can be localized or a manifestation of pregnancy, a general disorder, such as Addison's disease, or a side effect of various drugs, including antibiotics, antiarrhythmics, and antimalaria drugs. A common cause is inflammation or other skin injuries, including those related to acne vulgaris.

Hyperpigmentation may also result from exposure to sunlight or sunlight will darken already hyperpigmented areas. Solar lentigines or “age spots” are a common form a hyperpigmentation that may occur due to sun damage. These spots are often found on the face and hands or other areas that are frequently exposed to the sun. People with darker skin i.e. Asian, Mediterranean, Latinos/Hispanics, Native Americans, African, Pacific Islanders and those of Middle Eastern descent are more prone to hyperpigmentation, especially upon excess sun exposure. See Davis et al., Clin Aesthet Dermatol. 2010 July; 3(7): 20-31.

Facial hyperpigmentation is a broad term usually reflecting an increased amount of melanin in either the epidermis or the dermis, or both. Like hyperpigmentation in general, facial hyperpigmentation may be caused by many factors. Melasma is the most common cause of facial hyperpigmentation, but there are many other forms such as Riehl's melanosis, poikiloderma of Civatte, erythrose peribuccale pigmentaire of Brocq, erythromelanosis follicularis of the face and neck, linea fusca, and cosmetic hyperpigmentations. Perez-Bernal et al., Am J Clin Dermatol. 2000 September-October; 1 (5):261-8.

The term “melasma”, as used herein, refers to a light to dark brown, irregular hypermelanosis of the face and neck that is seen often in pregnant women. Melasma does not include the mucous membranes.

The term “postinflammatory hyperpigmentation” (PIH), as used herein, refers to the common sequelae of inflammatory dermatoses that is an acquired hypermelanosis occurring after cutaneous inflammation or injury. It can arise in all skin types, but tends to affect darker skinned patients with greater frequency and severity.

The term “lentigo” or “lentigines”, as used herein, refers to a small, sharply circumscribed, pigmented macule(s) surrounded by normal-appearing skin. Histologic findings may include hyperplasia of the epidermis and increased pigmentation of the basal layer. A variable number of melanocytes are present; these melanocytes may be increased in number, but they do not form nests (as in moles).

Melasma and other forms of facial or neck hyperpigmentation are cosmetically the most important. Treatment is challenging. Patients must try to avoid exposure to the sun or ultraviolet lamps and wear broad-spectrum sunscreens. There are a variety of topical depigmenting agents used to bleach the pigmented skin. For example, hydroquinone, tretinoin, azelaic acid, kojic acid, glycolic acid and licorice extract are use alone or in combination with other agents, with hydroquinone being the mainstay. Chemexfoliation and laser therapy may also be incorporated. However, these therapies have disadvantages. For instance, hydroquinone can cause contact dermatitis, nail discoloration, permanent leukoderma, and hypopigmentation of the surrounding normal skin that has been treated. Chemical peels can cause erythema, burning sensation, postinflammatory hyperpigmentation, reactivation of herpes simplex virus, superficial desquamation, and vesiculation. Lasers therapy can lead to complications such as dyschromias, blistering, and scars. See Davis et al., supra.

A particular object of the present invention is to provide S-acyl glutathione compositions having acyl groups to enhance skin penetration and transdermal absorption to improve the condition of the skin. The presence of the hydrocarbon chain of the apolar acyl group bonded to the glutathione thiol group enables the compounds of the invention to be effective as a topical application that can easily pass through the lipid bilayer of the cell membranes of epidermal and dermal cells. S-acyl glutathiones have lipophilic structures that make them fat soluble and able to pass through cell membranes and be absorbed directly into cells.

While not wishing to be bound by any theory, it is believed that palmitoyl groups in particular enhance the hydrophobicity and contribute to membrane association, similar to S-Palmitoylation observed with proteins. The association of the fatty acid chain is reversible (because the bond between palmitic acid and glutathione is a thio-ester bond) allowing the compound to be absorbed by the cell membranes.

S-acyl glutathione compounds of the present invention may be purchased or prepared by various means known to those of skill in the art. For example, enzymatic transthioesterification can be achieved by reacting glutathione with an appropriate acyl ester of coenzyme A (CoA) followed by purification from the water phase by HPLC or by chemically reacting glutathione with the corresponding acyl halide. See WO 2009/047728, supra, incorporated herein by reference. Another synthesis may be carried out by reacting the halide of the corresponding carboxylic acid with a solution of L-glutathione in trifluoroacetic acid under vacuum, adding ethyl acetate, and collecting the precipitated salt. See e.g. U.S. Pat. No. 3,984,569, supra, which is hereby incorporated by reference.

Topical compositions containing S-acyl glutathiones according to the present invention are intended to be topically applied to and absorbed by the skin tissue. While not wishing to be bound by any theory, the depigmenting effects of S-acyl glutathiones may result from (a) inactivation of the enzyme tyrosinase by binding with the copper-containing active site of the enzyme; (b) mediation of the switch mechanism from eumelanin to phaeomelanin production; (c) quenching of free radicals and peroxides that contribute to tyroosinase activation and melanin formation; or (d) modulation of depigmenting abilities of melanocytotoxic agents; similarly to the proposed mechanisms of glutathione. See Villarama et al., Intl J Cosmetic Sci 2005 27:147-153. Moreover, S-acyl glutathiones activate transketolase, increasing its activity by 300%, and prevent protein glycation and AGE formation. Therefore, after treatment for the recommended period of time, it is expected that decreased inflammation, irritation, and erythema of the skin will be observed, along with an increased skin elasticity and suppleness. Fine lines and wrinkles should be reduced and skin coloring should even out. The present invention thus is expected to treat hyperpigmentation, and particularly facial hyperpigmentation, while providing an overall improvement in skin appearance. Compositions and methods of the present invention are particularly preferred to treat melasma, PIH and lentigines.

Only effective amounts of topical compositions containing S-acyl glutathione are needed to achieve the aforementioned benefits and prevent typical effects of aging on the skin. Generally, topical application to skin tissue is accomplished in association with a dermatologically acceptable carrier, and particularly one in which the S-acyl glutathione is soluble per se or is effectively solubilized (e.g., as an emulsion or microemulsion). Where employed, the carrier is inert in the sense of not bringing about a deactivation or oxidation of the glutathione derived active ingredient(s), and in the sense of not bringing about any adverse effect on the skin areas to which it is applied.

In one preferred practice of the invention, one or more S-acyl glutathione derivatives is applied in admixture with the dermatologically acceptable carrier or vehicle (e.g., as a lotion, cream, ointment, soap, stick, or the like) so as to facilitate topical application and, in some cases, provide additional therapeutic effects as might be brought about, e.g., by moisturizing of the affected skin areas. While the carrier for the topical composition can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration and/or aid in the percutaneous delivery of the active agent(s). Many preparations are known in the art, and include lotions containing oils and/or alcohols and emollients vegetable oils, hydrocarbon oils and waxes, silicone oils, animal or marine fats or oils, glyceride derivatives, fatty acids or fatty acid esters, or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. In the preferred embodiment, the carrier is an oil in water emulsion.

As noted, these ingredients can be formulated into a cream, lotion, or gel, or a solid stick, by utilization of different proportions of the ingredients and/or by inclusion of thickening agents such as gums or other forms of hydrophilic colloids. One possible embodiment is a solution used to saturate a pad used to wipe affected areas; another is a cleanser; and others are lotions, creams, and gels, which are referred to herein as dermally or dermatologically acceptable carriers, and are formulated using conventional techniques known to those of ordinary skill in the art. In the most preferred embodiment, the ingredients are formulated into cream having a viscosity of 35,000 to 45,000 cps (measured on a Brookfield LVT Viscometer with a T/C spindle at 5 rpm) and a specific gravity of 0.9990 to 1.100.

The term “topical composition” as used herein shall mean the complete product including the S-acyl glutathione active ingredient, the carrier, and any adjuvants, thickeners, excipients, etc. as described herein which is applied to a person's skin.

The quantity of S-acyl glutathione active ingredient in the carrier may be varied or adjusted widely depending upon the particular application, the potency of the particular compound or the desired concentration. Generally, the quantity of S-acyl glutathione active ingredient will range between about 0.01% to about 20% by weight of the topical composition, more preferably, about 0.1% to about 10% by weight. In some applications, the quantity of S-acyl glutathione active ingredient will exceed 10% by weight. Generally, lower concentrations of S-acyl glutathione active ingredients in a carrier are suitable, depending upon the application regimen and the active and adjunct ingredients employed. In the most preferred embodiment, S-palmitoyl glutathione is present from about 3.00% to about 9.00% by weight.

Topical compositions containing S-acyl glutathione derivatives in admixture with the dermatologically acceptable carrier as described in this application may be used for the following methods: methods for the prevention and/or treatment of hyperpigmentation; methods of skin lightening; and methods of skin whitening.

Generally in the practice of methods of the invention, the topical composition is topically applied to the skin areas, such as that of the face, at predetermined intervals often as a moisturizer, lotion, or cream, it generally being the case that gradual improvement is noted with each successive application. Although immediate effects can be observed, enhanced results are observed when the topical composition is applied twice daily, preferably in the morning and evening. Insofar as has been determined based upon clinical studies to date, no adverse side effects are encountered. It is an advantage of the invention that compositions of the invention do not require a pharmaceutical prescription

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition.

Preservatives include, but are not limited to, C₁-C₃ alkyl parabens and phenoxyethanol, typically present in an amount ranging from about 0.1% to about 2.0% by weight percent, based on the total composition. A preferred preservative is ISP's Optiphen™ Plus, a liquid preservative formulation featuring a blend of phenoxyethanol, sorbic acid and an emollient base.

Emollients, typically present in amounts ranging from about 0.01% to 10% of the total composition include, but are not limited to, fatty esters, fatty alcohols, mineral oils, polyether siloxane copolymers, docosahexanoic acid (DHA) and mixtures thereof. Preferred emollients are Actiglow® (hydrolyzed glycosaminoglycans, propylene glycol, water, phenoxethanol) by Active Organics, squalane, shae butter, meadowfoam seed oil, isopropyl palmitate and DHA.

Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. A preferred humectant is shae butter.

Emulsifiers, typically present in amounts from about 1% to about 15% by weight of the composition, include, but are not limited to, stearic acid, cetyl alcohol, stearyl alcohol, steareth 2, steareth 20, acrylates/C10-30 alkyl acrylate crosspolymers, silicones, dimethylethanolamine (DMAE), phosphatidylcholine (PPC), docosahexanoic acid (DHA) and mixtures thereof. Preferred emulsifiers are sodium hyaluronate, Promulgen-D® (a mixture of 75% cetostearyl alcohol and 25% ethoxylate cetostearyl alcohol sold by Amerchol Corp.), Arlacel™ 165 (Glyceryl Stearate and PEG-100 Stearate sold by Croda Inc.) silicone (Dow Corning® 200 Fluid, 350 CST), dimethylaminoethanol, also known as DMAE, and Phospholipon® 90 G (phosphatidylcholine with 10% fatty acids sold by Phospholipid GmbH).

Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof.

Antioxidants, typically present in an amount ranging from about 0.01% to about 0.75% by weight of the composition, include, but are not limited to, butylated hydroxy toluene (BHT); vitamin C and/or vitamin C derivatives, such as fatty acid esters of ascorbic acid, particularly ascorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these. Particularly preferred antioxidants are those that provide additional benefits to the skin such as ascorbyl palmitate, sesame seed oil, lipoic acid, and Tocomin® 50 (palm oil, tocotrienols, tocopherol).

Buffering agents are employed in many compositions. Preferably, the amount of buffering agent is one that results in compositions having a pH ranging from about 4.0 to about 8.5, more preferably from about 4.5 to about 7.0, most preferably from about 5.0 to about 6.0. Typical buffering agents are chemically and physically stable agents commonly found in cosmetics, and can include compounds that are also adjunct ingredients such as citric acid, malic acid, and glycolic acid buffers.

Some embodiments of this invention contain at least one other adjunct ingredient in addition to S-acyl glutathione. Adjunct ingredients present in an amount ranging from 0.01°)/0 to about 20% by weight of the composition include, but are not limited to one or more of: isothiocyanates, caffeine, vitamin D3, lipoic acid; α-hydroxy acids such as glycolic acid or lactic acid; ascorbic acid and its derivatives, especially fatty acid esters of ascorbic acid; or tocotrienols and tocotrienol derivatives and vitamin E compositions enriched with tocotrienols or tocotrienol derivatives; and neuropeptides. Preferred adjunct agents include glycolic acid, citric acid, ascorbyl palmitate, Sepitonic™ M3 by Seppic, which contains magnesium aspartate, zinc gluconate and copper gluconate, Tocomin® 50, and Oligopeptide-17 and Oligopeptide-49.

Additional ingredients and methods as disclosed in my U.S. Pat. Nos. 5,376,361; 5,409,693; 5,545,398; 5,554,647; 5,574,063; 5,643,586; 5,709,868; 5,879,690; 6,191,121; 6,296,861; 6,437,004; and 6,979,459, which are hereby incorporated by reference, may also be used.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### EXAMPLES

Formulation: A formulation for an oil in water emulsion prepared by combing the following ingredients using conventional mixing techniques.

| Material | Wt. % |
|---|---|
| Purified Water | QS to 100 |
| S-acyl glutathione | 3.0-9.0 |
| Fatty acid derivatives of stearic acid | 5.0-10.0 |
| Isopropyl Palmitate | 1.0-5.0 |
| Tetrahexyldecyl ascorbate | 1.0-5.0 |
| Phosphatidylcholine | 1.0-5.0 |
| Hydrolyzed glycosaminoglycans | 0.5-3.5 |
| Mineral (magnesium, copper, zinc) salts | 0.5-1.5 |
| Squalane | 0.5-1.5 |
| Glycolic Acid | 0.5-1.5 |
| Sesame seed oil/meadowfoam seed oil | 0.5.-1.5 |
| Shea butter | 0.5.-1.5 |
| L-lipoic Acid | 0.25-0.75 |
| Penoxyethanol based preservatives | 0.25-0.75 |
| Dimethicone | 0.25-0.75 |
| Ascorbyl Palmitate | 0.25-0.75 |
| Disodium EDTA | 0.05-0.15 |
| Citric Acid | 0.05-0.15 |
| Fragrance | 0.0001-0.50 |

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A topical composition for treatment of hyperpigmentation, comprising:
an effective amount of S-acyl glutathione derivative of formula (I) 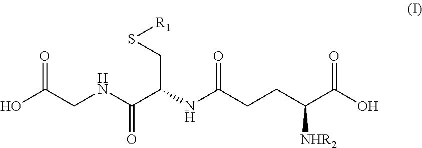
wherein R₁ consists of a saturated or unsaturated aliphatic C₁₂-C₂₄ group and R₂ is a hydrogen, aliphatic or aromatic acyl group; and
a dermatologically acceptable carrier.

2. The composition of claim 1, wherein R₁ is a C₁₆-C₂₀ group.

3. The composition of claim 2, wherein R₁ is a palmitoyl group.

4. The composition of claim 1, comprising from about 0.01% to about 20% by weight of S-acyl glutathione derivative.

5. The composition of claim 5, comprising from about 0.1% to about 10% by weight of S-acyl glutathione derivative.

6. The composition of claim 5, comprising from about 3.0% to about 9.0% by weight S-acyl glutathione derivative.

7. The composition of claim 1, wherein the carrier comprises fatty acid derivatives of stearic acid.

8. The composition of claim 1, further comprising one or more additional ingredients selected from the group consisting of: ascorbic acid and ascorbic acid derivatives, lipoic acid, α-hydroxy acids, and salts of magnesium, zinc and copper, and mixtures thereof.

9. A method for the treatment of hyperpigmentation comprising: applying a composition containing S-acyl glutathione derivative of formula (I) 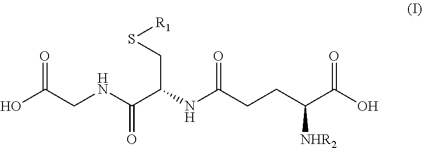
wherein R₁ consists of a saturated or unsaturated aliphatic C₁₂-C₂₄ group and R₂ is a hydrogen, aliphatic or aromatic acyl group; and a dermatologically acceptable carrier; to skin tissue.

10. The method of claim 9, wherein R₁ is a C₁₆-C₂₀ group.

11. The method of claim 10, wherein R₁ is a palmitoyl group.

12. The method of claim 9, wherein the composition comprises from about 0.01% to about 20% by weight of S-acyl glutathione derivative.

13. The method of claim 12, wherein the composition comprises from about 0.1% to about 10% by weight of S-acyl glutathione derivative.

14. The method of claim 13, wherein the composition comprises from about 3.0% to about 9.0% by weight of S-acyl glutathione derivative.

15. The method of claim 9, wherein the carrier comprises fatty acid derivatives of stearic acid.

16. The method of claim 9, wherein the composition further comprises one or more additional ingredients selected from the group consisting of: ascorbic acid and ascorbic acid derivatives, lipoic acid, α-hydroxy acids, and salts of magnesium, zinc and copper, and mixtures thereof.

17. The method of claim 9, wherein the hyperpigmentation comprises facial hyperpigmentation.

18. The method of claim 17, wherein the hyperpigmentation comprises at least one of melasma, postinflammatory hyperpigmentation and lentigines.

19. A method for skin lightening comprising applying a composition containing S-palmitoyl glutathione and a dermatologically acceptable carrier to skin tissue.

20. A method for skin whitening comprising applying a composition containing S-palmitoyl glutathione and a dermatologically acceptable carrier to skin tissue.
